(19) United States
(12) Patent Application Publication
(10) Pub. No.: US 2016/0299052 A1
(43) Pub. Date: Oct. 13, 2016

(54) METHODS AND SYSTEMS FOR TIME-OF-FLIGHT AFFINITY CYTOMETRY

(71) Applicant: ANCERA, INC., Branford, CT (US)

(72) Inventor: Hur KOSER, Branford, CT (US)

(21) Appl. No.: 14/777,511

(22) PCT Filed: Mar. 14, 2014
(86) PCT No.: PCT/US14/29376

§ 371 Date: Sep. 15, 2015

Related U.S. Application Data
(60) Provisional application No. 61/798,323, filed on Mar. 15, 2013.

(51) Int. Cl. G01N 15/10; G01N 33/543

(52) U.S. Cl. CPC G01N 15/1031; G01N 33/5438; G01N 2015/1006; G01N 2015/1075; G01N 27/128

(57) ABSTRACT

A device for determining the identity and concentration of target particles within a biocompatible ferrofluid medium is described. The system includes a fluidic channel that a sample of particles flow through; at least one magnetic field source configured to react repulsively with the particles; a channel wall with at least one receptor regions placed serially along the flow direction; and at least one thin electrode placed between the receptor regions to track changes in local impedance every time a target particle passes through the fluidic channel in close proximity.

### RELATED APPLICATIONS

This application claims benefit under 35 USC 119(e) of U.S. provisional patent application No. 61/798,323, filed Mar. 15, 2013, and entitled, “Time-of-flight Affinity Cytometry (TOFAC)” the entire disclosure of which is herein incorporated by reference in its entirety.

### FIELD OF THE DISCLOSURE

The present disclosure relates to identification in biocompatible ferrofluids and in particular, to systems and methods for identifying target particles in a biocompatible ferrofluid medium.

### BACKGROUND OF THE DISCLOSURE

Currently, there are at least two main approaches for determining the identity and concentration of a target biological species (from cellular down to molecular level). The first approach involves capturing or immobilizing such particles over a two-dimensional (2D) surface and subsequent generation of signals from moieties (tagged or label-free) captured. Traditional ELISA (enzyme-linked immunosorbent assay), optical scanners, and electronic bio-sensors, such as microcantilever sensors, functionalized nanowires or field-effect transistor sensors, all fall in this category. The second approach relies on flow cytometry, wherein target moieties are tagged with fluorescent antibodies and are scanned as they pass under an optical sensor one at a time. This enumeration could be done at the cellular level using fluorescently-tagged antibodies, or at the molecular level, using labels such as microbeads.

With both methods, antibodies or other receptors need to be incubated with the sample in order to immobilize them on a given surface, which takes time and is limited by diffusive mass transport. The kinetics of binding between the ligand and the target receptor is typically orders of magnitude faster than the diffusion-limited incubation, which currently dominates the duration of the assay.

Traditional 2D surface approaches also require considerable surface area covered with receptors in order to overcome diffusion limitations, thereby wasting valuable reagents. The diffusion limitations becomes a greater issue when there is laminar flow over the functionalized surface. In such cases, the faster the shear rate of flow over the functionalized surface, the larger the surface needs to be in order to capture target moieties. To that end, beyond a certain shear rate, moieties captured on the surface will be stripped away, thereby limiting the efficiency of capture/detection, as well as the speed of the assay.

The diffusion limitations of 2D surface approaches make flow cytometry a more attractive choice in assays involving large moieties with slow diffusion dynamics --for example, cellular assays—since free-floating tagged antibodies bind to cells much faster than the cells binding to antibodies immobilized on a surface. However, flow cytometry requires more sophisticated (and expensive) instrumentation to perform sample manipulation, fluid handling, high resolution fluorescent optical detection and sorting. Moreover, the number of optical channels is typically limited to a few and is determined by the complexity of the equipment and the availability of optical sources with different wavelengths.

### SUMMARY OF THE DISCLOSURE

Some embodiments of the present disclosure are a further application and development of previous series of disclosures, including, for example PCT publication no. WO2011/071912 and WO2012/057878, the noted disclosures of which are all herein incorporated by reference in their entireties.

In some embodiments, a system for identifying at least one target particle in a flow is provided, and may comprise at least one microfluidic channel having a wall, the channel configured to flow a plurality of particles in a first direction, at least one receptor region provided on the wall, where the at least one receptor region is functionalized with at least one first molecule for interacting with at least a first target particle contained in a flow of the plurality of particles. The system may also include a plurality of electrodes arranged before and after the receptor region, each electrode configured to signal a change in impedance proximate thereto upon the at least first target particle passing in proximity thereto. In some embodiments, a plurality of electrodes are positioned between receptor regions, and in some embodiments, a plurality of electrodes are positioned at each end of a receptor region.

In some embodiments, such systems may further comprise a processor configured to receive data corresponding to the changes in impedance as a result of particles passing in proximity to an electrode, and including computer instructions operating thereon configured to at least one of receive or otherwise obtain data corresponding to changes in impedance produced as a result of at least one first particle passing in proximity to one or more electrodes, at least tracking the at least one first target particle as it flows through the channel in the first direction based on the changes in impedance, and based on at least the tracking, determining the identity of the at least one first target particle.

In some embodiments, a method for identifying at least one target particle in a flow is provided which may be used with one and/or another of disclosed system embodiments. Such method embodiments may comprise flowing a plurality of particles containing at least one first target particle through at least one microfluidic channel, and monitoring changes in impedance in proximity to one or more of a plurality of electrodes, the impedance change produced as a result of at least one first particle passing in proximity to one or more electrodes. The method may also include at least tracking the at least one first target particle as it flows through the channel in the first direction based on the changes in impedance. Based on at least the tracking, the method may also determine the size and identity of the at least one first target particle from, for example, the amplitude and shape of the impedance signal, as well as particle velocity and changes to it.

One and/or another/various embodiments may additionally include one or more of the following features:
tracking comprises tracking at least a timing of the interaction of the first particles with the at least one receptor region;
interaction of the first target particles with a first receptor region affects at least one of the first target particle's drag and roll velocity along the channel;
the at least one receptor region comprises a plurality of receptor regions spaced apart along the wall, where each receptor region is functionalized with the at least one first molecule for interacting with the at least first target particle, and where the plurality of electrodes are arranged between the receptor regions;
each receptor region, where a plurality of regions are included, is functionalized with a different first molecule each configured to interact with different first target particle;
the electrodes comprise planar micro-electrodes;
the first target particle comprises a biological particle;
the first target particle comprises at least one of a bead, a molecule, a cell, a bacteria, a virus, DNA, RNA, a carbohydrate, a protein, a biomarker, a hormone, kinase, enzyme, cytokine, toxin, a bead functionalized with any of the foregoing, and any fragments of the foregoing;
the first molecule comprises a ligand;
each receptor region includes a diameter or length of between about 10 microns and about 1000 microns, or, between 10 microns and 500 microns, or, between 10 microns and 100 microns, or different ranges there between;
force means configured to exert a force, either directly or indirectly, on the at least one first target particles, such that the at least one first target particles are forced toward the at least one receptor region;
force means comprising at least one of gravity, hydrodynamic mean, dielectrophoretic means, electrostatic means and magnetic means;
a plurality of particles are provided within a ferrofluid medium and a force means is provided which may comprise a magnetic field means configured to apply a magnetic field to at least a portion of the channel, where the applied magnetic field applies an indirect force on at least the first target particles, such that they are directed toward the at least one receptor region, where the ferrofluid medium includes a plurality of magnetic particles;
the length or diameter of at least one receptor region is configured to result in at least 100 or greater interactions between the first target particles and the at least one receptor region;
the length or diameter of at least one receptor region is configured to result in at least 1000 or greater interactions between the first target particles and the at least one receptor region;
a strength of binding of the first molecules to the one receptor region is greater than a strength of binding of the first target particles to the first molecules;
the first molecule is bound to the at least one receptor region covalently;
an impedance frequency sensed by the electrodes for the changes in impedance in proximity thereto is between about 100 kHz and 10 MHz; and
data and/or signals generated by the electrodes include an amplitude of an impedance signal, and where computer instructions operating on a processor are configured to track the amplitude and correspond the amplitude to a size of the first target particles.

The above-noted embodiments, as well as other embodiments, will become even more evident with reference to the following detailed description and associated drawing, a brief description of which is provided below.

### BRIEF DESCRIPTION OF THE DRAWING

FIG. 1 illustrates a depiction of time-of-flight affinity cytometry according to some embodiments of the present disclosure.

FIG. 2 illustrates a time-of-flight affinity cytometry system.

### DETAILED DESCRIPTION OF SOME OF THE EMBODIMENTS

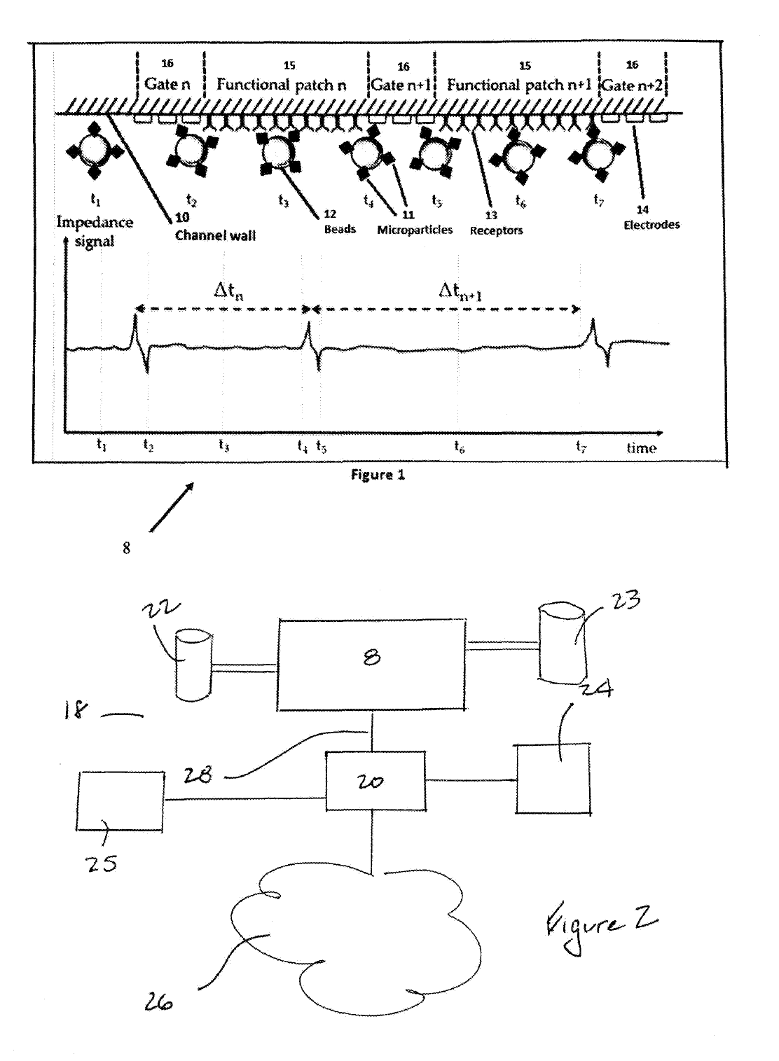

FIG. 1 illustrates some embodiments of the present disclosure, which are directed to a time-of-flight affinity cytometry 8 (“TOFAC”) illustrated schematically with a graph which depicts the signal strength of signals or information corresponding to the impedance change detected by the electrode(s) of at least one particle as it passes proximate to the electrode. In some embodiments, the identity of the particle(s) being tracked may be determined based on the tracking information. For example, a particle 11, e.g., a ligand, or a bead 12 having functionalized thereto the particle 11 on its surface, is directed through a force (e.g., either directly or indirectly on the particle) towards a wall 10 of at least one fluidic channel 9 of a system according to some embodiments of the present disclosure. The particles tracked may be biological particles including for example organic molecules, cells, moieties, and the like. Target particles may comprise any desired molecule or larger particle (e.g., a bead). In some embodiments, the particles may comprise any one or more of organic molecules, cells, bacteria, viruses, DNA, RNA, carbohydrates, proteins, peptides, biomarkers, hormones, kinases, enzymes, cytokines, toxins, a bead functionalized with any of the foregoing, and any fragment(s) of the foregoing.

At least one of the walls of the microfluidic channel include at least one receptor region, and in some embodiments, a plurality of regions spaced apart from one another are provided. Electrode(s) 14, which can be considered a gate region or Gate 16, e.g., “Gate n”, “Gate n+1”, etc., are positioned along the channel 12 to either side such regions, and may also be referred to as a function patch 15, e.g., “Functional patent n”, “Functional patch n+1”, etc. As the graph in FIG. 1 shows, a time period Δtₙ may represent a first time period of the particle that it takes to pass over a receptor area, i.e., between peaks of the signal amplitude of the respective electrode signal (e.g., before t₂ and ending between t₄ and t₅. As shown, t₁, a particle is received in the channel and approaches Gate n; t₂, the particle enters functional patch n, t₃, the particle is in the middle of functional patent n, t₄, the particle enters Gate n+1; at t₅, the particle enters functional patent n+1, at t₆, the particle is between Gate n+1 and Gate n+2, and at t₇, the particle enters Gate n+2. Δtₙ₊₁ represents the time period a particle spends in functional patent n+1.

Each such region, in some embodiments, may be functionalized with a molecule(s)(e.g., ligand) which are configured to bind or at the very least, interact with, a target particle, the resulting functionalized region comprising a receptor region. As noted, in some embodiments, a plurality of receptor regions may be provided along at least one of the walls of the at least one micro-channel (with an exception if a micro-channel is configured with a single wall, though a plurality of receptor regions may be placed thereon). Where a plurality of receptor regions are provided, each may be functionalized with a different molecule, where each specific molecule is configured to interact with different target particle.

Each receptor region may be sized with a diameter or length of between, for example, about 10 microns and about 1000 microns, or greater. For example, in some embodiments, the receptor regions size configuration is such that it can provide hundreds, and in some embodiments, thousands of interactions of the target particle along the wall of the micro-channel (thus, in some embodiments, hundreds, thousands of interactions with one or more receptor regions) as the particle rolls and/or skips along/over the receptor region on that wall.

In some embodiments, one or more, and preferably a plurality, of electrodes are arranged along the at least one wall of the micro-channel, with at least one positioned before and after each receptor region (or, in some embodiments, between), and each region having at least one to either end thereof. Each electrode may be configured to at least detect a change in impedance in proximity to the electrode, and one of ordinary skill in the art will recognize that the present disclosure includes the structure and means necessary to sense or otherwise detect such changes in impedance proximate each electrode (e.g., biasing/voltage means). The electrodes may comprise planar, micro-electrodes, or in other embodiments, may comprise other electronic components—such as field effect transistors or similar semiconducting devices—which are sensitive to changes in electric field patterns near their micro/nano-scale terminals.

In some embodiments, such means may include at least one processor which is configured with or otherwise operationally configured with an application program/computer instructions which enable the at least one processor to at least one of detect, monitor, and record impedance changes of at least one of the electrodes, and in some embodiments, each of the electrodes. For example, in some embodiments, the application/computer instructions may also be configured to receive or otherwise obtain data corresponding to the changes in impedance as a result of particles passing in proximity to an electrode. The data may include data associated with an amplitude of an impedance signal. An impedance frequency sensed by the electrodes for the changes in impedance in proximity thereto may be between, for example, about 100 kHz and 10 MHz.

The application/instructions may also be configured to at least track target particle (e.g., at least one first target particle) as it (or a plurality thereof) flows through the channel (e.g., in a first direction), the tracking being based on, in some embodiments, the changes in impedance. In some embodiments, based on at least the tracking, the application/instructions may be additionally configured to determine the identity of the at least one first target particle.

In some embodiments, the tracking of target particles may comprise tracking at least a timing of the interaction of the target particles with at least one receptor region, and in some embodiments, all receptor regions, based (again) on information determined by the change of impedance of electrodes bordering the receptor regions.

The interaction of target particles with a receptor region(s), according to some embodiments, affects at least one of the target particle's drag and roll velocity along the channel, such that its velocity along the channel slows down relative, for example, to other similarly sized particles contained in the flow, or relative to the flow in general. Changes in the drag/roll velocity of the a target particle may also be tracked as the particle traverses multiple receptor regions in sequence. Certain receptor regions in which the average travel velocity of the target particle is slower than in others may be labeled as “interacting”, and the amount of velocity reduction may be utilized to characterize and even quantify the interaction strength.

The force on the particles, and in particular, target particles, during a flow of particles along the micro-channel, may be at least one of a gravity force, a hydrodynamic force, a dielectrophoretic force, an electrostatic force and a magnetic force. The force may be a direct force on the particles, or specifically, on the target particles, or an indirect force on either thereof. With respect to embodiments including a magnetic force, such may be applied via a magnet, either or both of a permanent or electromagnet, or an electrode), and the particles may be suspended in a ferrofluid, which comprises a plurality of magnetic nanoparticles. In such embodiments, upon the application of a magnetic field by a source proximate to a wall of the fluidic channel, the magnetic nanoparticles are attracted to the stronger magnetic field regions near that wall, thereby indirectly displacing the non-magnetic particles (e.g., ligands, biological cells) and directing them away from magnetic field gradients toward the opposite wall of the fluidic channel. For more detailed information concerning ferrofluid-based magnetic systems for directing non-magnetic particles, see PCT publication nos. WO2011/071912 and WO2012/057878. Such disclosures teach that when a ferrofluid containing non-magnetic particles is exposed to a magnetic field, the resulting displacement of the non-magnetic particles away from the magnetic field sources is effectively a repulsive force acting on them.

Accordingly, when target molecules encounter a functionalized receptor region, they interact (e.g., partially and/or temporarily bind) with the functionalized molecules immobilized within that region. In some embodiments, the receptor molecules are configured to be more strongly immobilized on the channel surface than the strength of the interaction between the receptor and target molecules over the receptor region. In this way, according to some embodiments, the target particles can roll along each receptor region, binding and unbinding with the functionalized molecules, such that the target particle still flows along the wall (and/or channel), but is slowed down relative to the general flow. Moreover, the functionalized molecules are configured to be bound to the surface of the wall such that the a target particle preferably cannot “rip” the molecule from the receptor region. To this end, the functionalized molecule may be bound to a receptor region either covalently or through a strong non-covalent interaction, such as avidin/streptavidin-biotin binding mechanism.

Time signatures of target particles passing in proximity to an electrode may be at least one of tracked, recorded, and analyzed, and result, at least partially from, the roll of the target particle(s), for example, over/through each receptor region. Accordingly, with a constant shear flow rate over the surface of the wall of the micro-channel, the average travel speed of the target particle varies due to, in some embodiments primarily due to, interactions between the molecules functionalized to the receptor region. A greater level of interaction within a given receptor region may correspond to measurable delays in transport through that region (see FIG. 1, functional patch n+1), which may be referred to as a “time-of-flight” measurement. This measurement may enable a characterization of affinity between the target particles and the various receptors. To that end, a longer travel time through a receptor region(s), may correspond to a larger degree of affinity (i.e., a larger association constant).

FIG. 2 represents a time-of-flight affinity cytometry system 18 according to some embodiments. In such embodiments, for example, the TOFAC system 8 according to FIG. 1, and in some embodiments, a plurality of such systems, or a plurality of fluidic channels as exemplified in FIG. 1, each with electrodes and functionalized regions, is connected with or otherwise in communication with other components, including one and/or more of the following:
at least one computer or computer processor 20 operational with at least one of an application, computer code/instruction, for carrying our various processes of the system to at least one of input, control, operate, change, adjust, output, track at least one type(s) of particle(s), and determine or otherwise identify at least one type(s) of particle;
a reservoir 22 or holding area for supplying a flow of particles to the TOFAC channel, via, for example, an inlet or inlet portion;
an outlet 23 or holding area for receiving the particles (and/or the remainder of the flow) after passing through the TOFAC 8;
a database 25 in communication with the computer, the database containing data and/or code configured for use in input, control, operate, change, adjust, output, track at least one type(s) of particle(s), and determine or otherwise identify at least one type(s) of particle;
an input and/or output means configured to input commands and/or data into the processor; and
communication means (see below) for communicating with a remote computer, database and/or mobile device via, for example, a network 26.

Some embodiments of the present disclosure include communication means (e.g., reference number 28, FIG. 2) between a computing system or device, which may be used to at least one of operate, control, track, record, analyze, process and output data from the flow of particles, and in particular, target particles, and any other components necessary to carry out the objects and/or advantages of the system. In some embodiments, the communication can be wired and provided through electrical connections. In some embodiments, the communication can be wireless via an analog short range communication mode, or a digital communication mode including WIFI or BLUETOOTH®. Additional examples of such communication can include a network. The network can include a local area network (“LAN”), a wide area network (“WAN”), or a global network, for example. The network can be part of, and/or can include any suitable networking system, such as the Internet, for example, and/or an Intranet. Generally, the term “Internet” may refer to the worldwide collection of networks, gateways, routers, and computers that use Transmission Control Protocol/Internet Protocol (“TCP/IP”) and/or other packet based protocols to communicate therebetween.

In some embodiments, one or more systems may include a single or plurality of transmission elements for communication between components thereof. In some embodiments, the transmission element can include at least one of the following: a wireless transponder, or a radio-frequency identification (“RFID”) device. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to-digital (“A/D”) converter, and/or an electrical circuit for analog or digital short range communication.

In some embodiments, any relevant components of TOFAC system embodiments can include one or more processors, one or more memories, one or more storage devices, and at least one input and/or output device.

Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof. These various implementations, such as associated with the disclosed TOFAC systems and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., non-transitory mediums including, for example, magnetic discs, optical disks, flash memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smartphone, media player or personal data assistant (“PDA”). Other kinds of devices may be used to provide for interaction with a user as well. For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements/features from any other disclosed methods, systems, and devices, including any and all features corresponding to cytometry and the like. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure). Likewise, some of the embodiments of the disclosure may be patentably distinct from the prior art by not having one or more specific prior art elements/features. In other words, claims to some embodiments are patentably distinct over the prior art by reciting lack of one or more elements/features (i.e., negative limitations).

1. A system for identifying at least one target particle in a flow comprising:
at least one microfluidic channel having a wall, the channel configured to flow a plurality of particles in a first direction;
at least one receptor region provided on the wall, wherein the at least one receptor region is functionalized with at least one first molecule for interacting with at least a first target particle contained in a flow of the plurality of particles; and
a plurality of electrodes arranged before and after the receptor region, each electrode configured to signal a change in impedance proximate thereto upon the at least first target particle passing in proximity thereto.

2. The system of claim 1, further comprising a processor configured to receive data corresponding to the changes in impedance as a result of particles passing in proximity to an electrode, and including computer instructions operating thereon configured to:
receive or otherwise obtain data corresponding to changes in impedance produced as a result of at least one first particle passing in proximity to one or more electrodes;
at least track the at least one first target particle as it flows through the channel in the first direction based on the changes in impedance, and
based on at least the tracking, determine an identity of the at least one first target particle.

3. The system of claim 2, wherein the tracking comprises tracking at least a timing of the interaction of the first particles with the at least one receptor region.

4. The system of claim 1, wherein the interaction of the first target particles with a first receptor region affects at least one of the first target particle's drag and roll velocity along the portion of the channel that contains the receptor region.

5. The system of claim 1, wherein the at least one receptor region comprises a plurality of receptor regions spaced apart along the wall, wherein each receptor region is functionalized with the at least one first molecule for interacting with the at least first target particle, and wherein the plurality of electrodes are arranged between the receptor regions.

6. The system of claim 5, wherein each receptor region is functionalized with a different first molecule, wherein each different first molecule is configured to interact with a different first target particle.

7. The system of claim 1, wherein the the plurality of electrodes comprise planar micro-electrodes.

8. The system of claim 1, further comprising a sensor region, wherein the sensor region comprises a semiconductor device configured to respond to localized changes in electric field patterns over its surface.

9. The system of claim 8, wherein the semiconductor device comprises a field effect transistor.

10. The system of claim 1, wherein the first target particle comprises a biological particle.

11. The system of claim 1, wherein the first target particle comprises at least one of a bead, a molecule, a cell, a bacteria, a virus, DNA, RNA, a carbohydrate, a protein, a biomarker, a hormones, kinases, enzymes, cytokines, toxins, a bead functionalized with any of the foregoing, and any fragments of the foregoing.

12. The system of claim 1, wherein the first molecule comprises a ligand.

13. The system of claim 1, wherein each receptor region includes a diameter or length of between about 10 microns and about 100 microns.

14. The system of claim 1, further comprising force means configured to exert a force, either directly or indirectly, on the at least one first target particles, such that the at least one first target particles are forced toward the at least one receptor region.

15. The system of claim 14, wherein the force means comprises at least one of gravity, hydrodynamic means, dielectrophoretic means, electrostatic means and magnetic means.

16. The system of claim 14, wherein the plurality of particles are provided within a ferrofluid medium and the force means comprises a magnetic field means configured to apply a magnetic field to at least a portion of the channel, wherein the applied magnetic field applies an indirect force on at least the first target particles, such that they are directed toward the at least one receptor region.

17. The system of claim 16, wherein the ferrofluid medium includes a plurality of magnetic nanoparticles.

18. The system of claim 1, wherein the length or diameter of at least one receptor region is configured to result in at least 100 or greater interactions between the first target particles and the at least one receptor region.

19. The system of claim 1, wherein the length or diameter of at least one receptor region is configured to result in at least 1000 or greater interactions between the first target particles and the at least one receptor region.

20. The system of claim 1, wherein the strength of binding of the first molecules to the receptor region is greater than a strength of binding of the first target particles to the first molecules.

21. The system of claim 1, wherein the first molecule is bound to the at least one receptor region covalently.

22. The system of claim 1, wherein an impedance frequency sensed by the electrodes for the changes in impedance in proximity thereto is between about 100 kHz and about 10 MHz.

23. The system of claim 2, wherein the data includes an amplitude of an impedance signal, and wherein the computer instructions are further configured to track the amplitude and correspond the amplitude to a size of the first target particles.

24. A system for identifying at least one target particle in a flow comprising:
at least one microfluidic channel having a wall, the channel configured to flow a plurality of particles in a first direction;
at least one receptor region provided on the wall, wherein the at least one receptor region is functionalized with at least one first molecule for interacting with at least a first target particle contained in a flow of the plurality of particles;
a plurality of electrodes arranged before and after the receptor region, each electrode configured to signal a change in impedance proximate thereto upon the at least first target particle passing in proximity thereto; and
a processor configured to receive data corresponding to the changes in impedance as a result of particles passing in proximity to an electrode, and including computer instructions operating thereon configured to:
receive or otherwise obtain data corresponding to changes in impedance produced as a result of at least one first target particle passing in proximity to one or more electrodes;
at least track the at least one first target particle as it flows through the channel in the first direction based on the changes in impedance, and
based on at least the tracking, determine an identity of the at least one first target particle.

25-42. (canceled)
